SEQUENCE LISTING (28 sequences)

SEQ ID NO: 1 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRNNWPLTFGGGTKVEIK

SEQ ID NO: 2 - protein, 124 aa, Homo sapiens
EVQLVESGGGLVQPGGSLRLSCAASGFTFSSYDMHWVRQATGKGLEWVSGITTAGDTYYPGSVKGRFTISRENAKNSLYLQMNSLRAGDTAVYYCARGRISMDRGVKNNWFDPWGQGTLVTVSS

SEQ ID NO: 3 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 4 - protein, 124 aa, Homo sapiens
EVQLVESGGGLVRPGGSLRLSCAASGFTFSNYDMHWVRQATGKGLEWVSAITAAGDIYYPGSVKGRFTISRENAKNSLYLQMNSLRAGDTAVYYCARGRYSGSGSYYNDWFDPWGQGTLVTVSS

SEQ ID NO: 5 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYHSYPLTFGGGTKVEIK

SEQ ID NO: 6 - protein, 128 aa, Homo sapiens
QVQLVESGGGVVQPGRSLRLSCAASGFTFSSYGMHWVRQAPGEGLEWVAVIWYDGTFKYYADSVKGRFTISRDNSKNTLSLLMNSLRAEDTAVYYCTRGGYYGSGSSFDYYYYGMDVWGQGTTVTVSS

SEQ ID NO: 7 - protein, 107 aa, Homo sapiens; identical to SEQ ID NO: 5

SEQ ID NO: 8 - protein, 117 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDDLDYFDYWGQGTQVTVSS

SEQ ID NO: 9 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 10 - protein, 117 aa, Homo sapiens; identical to SEQ ID NO: 8

SEQ ID NO: 11 - protein, 108 aa, Homo sapiens; identical to SEQ ID NO: 9

SEQ ID NO: 12 - protein, 128 aa, Homo sapiens; identical to SEQ ID NO: 6

SEQ ID NO: 13 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 14 - protein, 124 aa, Homo sapiens
EVQLVESGGGLVQPGGSLRLSCAASGFTFSSYDMHWVRQATGKGLEWVSAISTAGDTYYPGSVKGRFTISRENAKNSLYLQMNSLRVGDTAVYYCARGRFDGSGSYYNDWFDPWGQGTLVTVSS

SEQ ID NO: 15 - protein, 107 aa, Homo sapiens
AIQLTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 16 - protein, 124 aa, Homo sapiens; identical to SEQ ID NO: 14

SEQ ID NO: 17 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRYNWPLTFGGGTKVEIK

SEQ ID NO: 18 - protein, 124 aa, Homo sapiens; identical to SEQ ID NO: 14

SEQ ID NO: 19 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 20 - protein, 119 aa, Homo sapiens
QVQLVESGGGVVQPGRSLRLSCAATGFTFSSYGMHWVRQAPGKGLEWVAVIWYDGSKKYYTDSVKGRFTISRDNSKNTLYLQMNSLRAEDTAVYYCARDQNWIDVFDIWGQGTMVTVSS

SEQ ID NO: 21 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 22 - protein, 124 aa, Homo sapiens
QPQLVQSGAEVKKPGASVKVSCKVSGNTLTELSMHWVRQAPGKGLEWMGGFDPENGEAIYAQKFQGRVTMTADTSTDTAYMDLSSLRSEDTAVYYCATDLAGGSDFYYYGLDVWGQGTTVTVSS

SEQ ID NO: 23 - protein, 107 aa, Homo sapiens [sequence omitted]

SEQ ID NO: 24 - protein, 330 aa, Homo sapiens [sequence omitted]

SEQ ID NO: 25 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2014/0093512 A1
(43) Pub. Date: Apr. 3, 2014

(54) PHARMACEUTICAL FORMULATION COMPRISING AN ANTIBODY AGAINST P-SELECTIN AND A SUGAR SELECTED FROM SUCROSE AND TREHALOSE

(71) Applicant: Hoffmann-La Roche Inc., Nutley, NJ (US)

(72) Inventors: Ulla Grauschopf, Riehen (CH); Hanns-Christian Mahler, Basel (CH); Oliver Boris Stauch, Mountain View, CA (US)

(73) Assignee: Hoffmann-La Roche Inc., Nutley, NJ (US)

(21) Appl. No.: 14/031,199

(22) Filed: Sep. 19, 2013

Related U.S. Application Data
(63) Continuation of application No. 13/854,254, filed on Apr. 1, 2013.
Application No. 13/854,254 is a continuation of application No. 13/561,233, filed on Jul. 30, 2012, now abandoned.
Application No. 13/561,233 is a continuation of application No. 12/559,555, filed on Sep. 15, 2009, now abandoned.

(30) Foreign Application Priority Data
Sep. 19, 2008 (EP) 08164746.3

(51) Int. Cl. A61K 39/395; C07K 16/18

(52) U.S. Cl. CPC A61K 39/3955; C07K 16/18
USPC 424/142.1; 424/172.1

(57) ABSTRACT

This invention relates to a pharmaceutical formulation of an antibody against P-Selectin, a process for the preparation of the formulation and uses of the formulation.

### PRIORITY TO RELATED APPLICATIONS

This application is a continuation of U.S. application Ser. No. 13/561,233, filed Jul. 30, 2012, which is a continuation of U.S. application Ser. No. 12/559,555, filed Sep. 15, 2009, which claims the benefit of European Patent Application No. 08164746.3, filed Sep. 19, 2008. The entire contents of the above-identified applications are hereby incorporated by reference.

### BACKGROUND OF THE INVENTION

Antibodies against P-Selectin are known from, e.g., U.S. Pat. No. 4,783,399, WO 93/06863, Geng et al (J. Biol. Chem., 266 (1991) 22313-22318), WO 93/21956 and WO 2005/100402. Exemplary antibodies against P-Selectin are described in WO 2005/100402 and include anti-bodies which are characterized in that the variable heavy chain amino acid sequence CDR3 of said antibody is selected from the group consisting of the heavy chain CDR3 sequences SEQ ID NO: 38, 39, 40, 41 or 42.

### SUMMARY OF THE INVENTION

The present invention relates to a pharmaceutical formulation of an antibody against P-Selectin, a process for the preparation and uses of the formulation.

### DETAILED DESCRIPTION OF THE INVENTION

In a first aspect, the invention relates to a pharmaceutical formulation having a pH in the range of 4.0 to 7.0 comprising:

1 to 200 mg/mL of an antibody against P-selectin;

1 to 100 mM of a buffer;

0.001 to 1% of a surfactant; and

a component selected from the group consisting of
(a) 10 to 500 mM of a stabilizer;
(b) 10 to 500 mM of a stabilizer and 5 to 500 mM of a tonicity agent; and
(c) 5 to 500 mM of a tonicity agent.

The formulation according to the invention can be in a liquid form, a lyophilized form or in a liquid form reconstituted from a lyophilized form.

Preferred antibodies are characterized in

i) comprising a variable heavy chain and a variable light chain, characterized in that the variable heavy chain comprises CDR sequences CDR1, CDR2 and CDR3 and CDR1 being selected from the group consisting of SEQ ID NOs: 29, 30, 31, 32, CDR2 being selected from the group consisting of SEQ ID NOs: 33, 34, 35, 36, 37, CDR3 being selected from the group consisting of SEQ ID NOs: 38, 39, 40, 41, 42, wherein said CDRs are selected independently of each other,
ii) that the variable light chain comprises CDR sequences CDR1, CDR2 and CDR3, and CDR1 is selected from SEQ ID NOs: 43, 44, CDR2 is selected from SEQ ID NOs: 45, 46 and CDR3 is selected from SEQ ID NOs: 47, 48, 49, 50, 51, 52 wherein said CDRs are selected independently of each other,
iii) containing as heavy chain CDRs the CDRs of SEQ ID NO: 2 and as light chain CDRs the CDRs of SEQ ID NO: 1, as heavy chain CDRs the CDRs of SEQ ID NO: 4 and as light chain CDRs the CDRs of SEQ ID NO: 3, as heavy chain CDRs the CDRs of SEQ ID NO: 6 and as light chain CDRs the CDRs of SEQ ID NO: 5, as heavy chain CDRs the CDRs of SEQ ID NO: 8 and as light chain CDRs the CDRs of SEQ ID NO: 7, as heavy chain CDRs the CDRs of SEQ ID NO: 10 and as light chain CDRs the CDRs of SEQ ID NO: 9, as heavy chain CDRs the CDRs of SEQ ID NO: 12 and as light chain CDRs the CDRs of SEQ ID NO: 11, as heavy chain CDRs the CDRs of SEQ ID NO: 14 and as light chain CDRs the CDRs of SEQ ID NO: 13, as heavy chain CDRs the CDRs of SEQ ID NO: 16 and as light chain CDRs the CDRs of SEQ ID NO: 15, as heavy chain CDRs the CDRs of SEQ ID NO: 18 and as light chain CDRs the CDRs of SEQ ID NO: 17, as heavy chain CDRs the CDRs of SEQ ID NO: 20 and as light chain CDRs the CDRs of SEQ ID NO: 19, or as heavy chain CDRs the CDRs of SEQ ID NO: 22 and as light chain CDRs the CDRs of SEQ ID NO: 21,
iv) that said antibody binds P-selectin and comprises a variable heavy and light region independently selected from the group consisting of the heavy chain variable domain defined by amino acid sequence SEQ ID NO:2 and the light chain variable domain defined by SEQ ID NO:1; the heavy chain variable domain defined by amino acid sequence SEQ ID NO:4 and the light chain variable domain defined by SEQ ID NO:3; the heavy chain variable domain defined by amino acid sequence SEQ ID NO:6 and the light chain variable domain defined by SEQ ID NO:5; the heavy chain variable domain defined by amino acid sequence SEQ ID NO:8 and the light chain variable domain defined by SEQ ID NO:7; the heavy chain variable domain defined by amino acid sequence SEQ ID NO:10 and the light chain variable domain defined by SEQ ID NO:9; the heavy chain variable domain defined by amino acid sequence SEQ ID NO:12 and the light chain variable domain defined by SEQ ID NO:11; the heavy chain variable domain defined by amino acid sequence SEQ ID NO:14 and the light chain variable domain defined by SEQ ID NO:13; the heavy chain variable domain defined by amino acid sequence SEQ ID NO:16 and the light chain variable domain defined by SEQ ID NO:15; the heavy chain variable domain defined by amino acid sequence SEQ ID NO:18 and the light chain variable domain defined by SEQ ID NO:17; the heavy chain variable domain defined by amino acid sequence SEQ ID NO:20 and the light chain variable domain defined by SEQ ID NO:19; the heavy chain variable domain defined by amino acid sequence SEQ ID NO:22 and the light chain variable domain defined by SEQ ID NO:21,
v) that the heavy chain variable region comprises an amino acid sequence independently selected from the group consisting of SEQ ID NO: 2, 4, 6, 8, 10, 12, 14, 16, 18, 20 and 22,
vi) that the light chain variable region comprises an amino acid sequence independently selected from the group consisting of SEQ ID NO: 1, 3, 5, 7, 9, 11, 13, 15, 17, 19 and 21.

The CDR sequences can be determined according to the standard definition of Kabat et al., Sequences of Proteins of Immunological Interest, 5th ed., Public Health Service, National Institutes of Health, Bethesda, Md. (1991). CDRs on each chain are separated by framework amino acids. CDRs of SEQ ID NO: 1-22 are shown in SEQ ID NO: 29-52.

In one embodiment, the antibody is characterized in binding P-selectin and does not bind complement factor C1q and/or Fc receptor. These antibodies do not elicit the complement dependent cytotoxicity (CDC) and/or antibody-dependent cellular cytotoxicity (ADCC).

Preferably, this antibody is characterized in that it binds P-selectin, contains a Fc part derived from human origin and does not bind complement factor C1q. More preferably, this antibody is a human or humanized antibody.

In another embodiment, the antibody is characterized in that the constant chains are of human origin. Such constant chains are well known in the state of the art and e.g. described by Kabat (see e.g. Johnson and Wu, Nucleic Acids Res. 28 (2000) 214-218). For example, a useful human heavy chain constant region comprises an amino acid sequence independently selected from the group consisting of SEQ ID NO: 24, 25, 26, 27 and 28. For example, a useful human light chain constant region comprises an amino acid sequence of a kappa-light chain constant region of SEQ ID NO: 23.

The term “binding to P-selectin” as used herein means the binding of the antibody to P-selectin in either a BIAcore® assay (Pharmacia Biosensor AB, Uppsala, Sweden) or in an ELISA in which either purified P-selectin or P-selectin CHO transfectants are coated onto microtiter plates.

In the BIAcore® assay, the antibody is bound to a surface and binding of P-selectin is measured by Surface Plasmon Resonance (SPR). The affinity of the binding is defined by the terms ka (rate constant for the association of the antibody from the antibody/antigen complex), kd (dissociation constant), and K_{D} (kd/ka). In further another embodiment, the antibodies show a K_{D} of 10⁻⁸ or less, preferably of about 10⁻¹¹ to 10⁻⁹ M (see examples). Accordingly, preferred antibody is that as described above, wherein the antibody bind to P-selectin with a K_{D} value of less than 10⁻⁸ M in a BIAcore® assay, preferably wherein the K_{D} range is 10⁻¹¹ to 10⁻⁹ M.

Preferably, the antibody is of IgG1 or IgG4 human subtype.

More preferably, the antibody is characterized in that the antibody is an antibody of human subclass IgG1, containing at least one mutation in L234, L235, D270, N297, E318, K320, K322, P331 and/or P329 or an antibody of human subclass IgG4, containing at least one mutation in L235 and 5228 (numbering according to EU index).

In the P-selectin-specific ELISA purified P-selectin is coated onto microtiter plates and the binding of the antibody to P-selectin is detected with a biotinylated anti-human IgG and the usual steps of an ELISA. The EC₅₀ values in this assay range preferably between 0.002 and 0.03 μg/ml on P-selectin CHO cells, i.e. preferred antibodies are those, wherein the EC50 values for P-selectin binding are in the range of 0.002 to 0.03 μg/ml on P-selectin presenting CHO cells in an ELISA assay. In an assay in which P-selectin expressing CHO transfectants are coated onto the microtiter plate, the EC50 values range between 0.01 and 0.08 μg/ml, preferably between 0.01 and 0.04 μg/ml.

EC₅₀ values on E- and L-selectin transfectants are preferably above 100 μg/ml. The preferred antibodies are characterized in that they bind at least 1000 fold more specifically to P-selectin than to E- and/or L-selectin as measured by EC₅₀ values in an ELISA assay, wherein P- and E- and/or L-selectin are coated onto the microtiter plate.

The antibodies are preferably capable of binding to P-selectin in the presence of the P-selectin fragment aa 60-75 (Swiss-Prot sequence P16109) and/or do not competitively inhibit the binding of an antibody secreted by a cell line designated ATCC Accession No. HB11041 to P-selectin.

The antibodies preferably do not inhibit the interaction of P-selectin with platelet membrane glycoprotein GPIbα in an ELISA assay format. In the ELISA glycocalicin, the soluble extracellular portion of GPIbα was immobilized on the wells of microtiter plates, as described (Romo et al., J Exp Med 190:803 (1999), and the binding of purified P-selectin after preincubation with the P-selectin HuMabs was detected with a polyclonal anti-P-selectin antibody.

The preferred antibody is characterized in that it does not bind the C3 protein, more preferably it is characterized in that it does not elicit complement-dependent cytotoxicity (CDC). Further, the antibody may be characterized it does not bind to Fey receptors on NK effector cells. Preferably, the antibody is characterized that it is an antibody of human subclass IgG 1, containing at least one mutation in L234, L235, D270, N297, E318, K320, K322, P331 and/or P329 or an antibody of human subclass IgG4, containing at least one mutation in L235 and S228 (numbering according to EU index). The preferred antibody is characterized in that it does not elicit antibody-dependent cellular cytotoxicity (ADCC).

The more preferred antibodies are characterized in that they bind P-selectin and that they comprise a variable region independently selected from the group consisting of the light chain variable domain defined by amino acid sequence SEQ ID NO:1 and the heavy chain variable domain defined by SEQ ID NO:2; the light chain variable domain defined by amino acid sequence SEQ ID NO:3 and the heavy chain variable domain defined by SEQ ID NO:4; the light chain variable domain defined by amino acid sequence SEQ ID NO:5 and the heavy chain variable domain defined by SEQ ID NO:6; the light chain variable domain defined by amino acid sequence SEQ ID NO:7 and the heavy chain variable domain defined by SEQ ID NO:8; the light chain variable domain defined by amino acid sequence SEQ ID NO:9 and the heavy chain variable domain defined by SEQ ID NO:10; the light chain variable domain defined by amino acid sequence SEQ ID NO:11 and the heavy chain variable domain defined by SEQ ID NO:12; the light chain variable domain defined by amino acid sequence SEQ ID NO:13 and the heavy chain variable domain defined by SEQ ID NO:14; the light chain variable domain defined by amino acid sequence SEQ ID NO:15 and the heavy chain variable domain defined by SEQ ID NO:16; the light chain variable domain defined by amino acid sequence SEQ ID NO:17 and the heavy chain variable domain defined by SEQ ID NO:18; the light chain variable domain defined by amino acid sequence SEQ ID NO:19 and the heavy chain variable domain defined by SEQ ID NO:20; and the light chain variable domain defined by amino acid sequence SEQ ID NO:21 and the heavy chain variable domain defined by SEQ ID NO:22.

Preferably, the antibodies comprise the light chain variable domain defined by amino acid sequence SEQ ID NO:3 and the heavy chain variable domain defined by SEQ ID NO:4.

The preferred antibodies are characterized in that the antibodies are of human IgG4 subclass or comprise at least one amino acid mutation causing non-binding to complement factor C1q. These variant antibodies comprise for example the amino acid sequence independently selected from the group consisting of SEQ ID NO: 25 or SEQ ID NO:26 and SEQ ID NO:28.

A “variant” anti-P-selectin antibody, refers herein to a molecule which differs in amino acid sequence from a “parent” anti-P-selectin antibody amino acid sequence by virtue of addition, deletion and/or substitution of one or more amino acid residue(s) in the parent antibody sequence. Preferably, the variant comprises one or more amino acid substitution(s) in one or more constant or variable region(s) of the parent antibody, more preferably in the constant region. For example, the variant may comprise at least one, e.g. from about one to about ten, and preferably from about two to about five, substitutions in one or more variable regions of the parent antibody. Ordinarily, the variant will have an amino acid sequence having at least 90% amino acid sequence identity with the parent antibody constant and/or variable domain sequences, more preferably at least 95%, and most preferably at least 99%.

Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind human P-selectin and preferably has properties, which are superior to those of the parent antibody. For example, the variant may have a stronger binding affinity, enhanced ability to treat a disease associated with critical limb ischemia or peripheral arterial occlusive disease (CLI/PAOD).

The variant antibody of particular interest herein is one which displays at least about 4 fold, enhancement in inhibitory activity in the adhesion assay when compared to the parent antibody because of the elimination of the binding to the Fcγ receptors.

The “parent” antibody herein is one, which is encoded by an amino acid sequence used for the preparation of the variant. Preferably, the parent antibody has a human framework region and, if present, has human antibody constant region(s). For example, the parent antibody may be a humanized or human antibody.

The antibodies according to the invention include, in addition, such antibodies having “conservative sequence modifications”, nucleotide and amino acid sequence modifications, which do not affect or alter the above-mentioned characteristics of the antibody according to the invention. Modifications can be introduced by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (es., threonine, valine, isoleucine) and aromatic side chains (e.g., tyrosine, phenylalanine, tryptophan, histidine). Thus, a predicted nonessential amino acid residue in a human anti-P-selectin antibody can be preferably replaced with another amino acid residue from the same side chain family.

Amino acid substitutions can be performed by mutagenesis based upon molecular modeling as described by Riechmann et al., Nature 332 (1988) 323-327 and Queen et al., Proc. Natl. Acad. Sci. USA 86 (1989) 10029-10033.

Further preferred antibodies comprise an K-light chain constant region as defined by SEQ ID NO:23.

Further preferred antibodies are those defined as IgG1v1 (PVA-236; GLPSS331 as specified by E233P; L234V; L235A; delta G236; A327G; A330S; P331S), IgG1v2 (L234A; L235A) and IgG4v1 (S228P; L235E).

Description of the sequence listing

SEQ ID NO:1 LC 1004-001 light chain, variable domain of HuMab 1004-001
SEQ ID NO:2 LC1004-001 heavy chain, variable domain of HuMab 1004-001
SEQ ID NO:3 LC 1004-002 light chain, variable domain of HuMab 002
SEQ ID NO:4 LC 1004-002 heavy chain, variable domain of HuMab 002
SEQ ID NO:5 LC 1004-003 light chain, variable domain of HuMab 003
SEQ ID NO:6 LC 1004-003 heavy chain, variable domain of HuMab 003
SEQ ID NO:7 LC 1004-004 light chain (I), variable domain of HuMab 004 (I)
SEQ ID NO:8 LC 1004-004 heavy chain (I), variable domain of HuMab 004 (I)
SEQ ID NO:9 LC 1004-004 light chain (II), variable domain of HuMab 004 (II)
SEQ ID NO:10 LC 1004-004 heavy chain (II), variable domain of HuMab 004 (II)
SEQ ID NO:11 Light chain, variable domain of HuMab 005
SEQ ID NO:12 Heavy chain, variable domain of HuMab 005
SEQ ID NO:13 Light chain, variable domain of HuMab 010 (I)
SEQ ID NO:14 Heavy chain, variable domain of HuMab 010 (I)
SEQ ID NO:15 Light chain, variable domain of HuMab 010 (II)
SEQ ID NO:16 Heavy chain, variable domain of HuMab 010 (II)
SEQ ID NO:17 Light chain, variable domain of HuMab 010 (III)
SEQ ID NO:18 Heavy chain, variable domain of HuMab 010 (HI)
SEQ ID NO:19 Light chain, variable domain of HuMab 011
SEQ ID NO:20 Heavy chain, variable domain of HuMab 011
SEQ ID NO:21 Light chain, variable domain of HuMab 017
SEQ ID NO:22 Heavy chain, variable domain of HuMab 017
SEQ ID NO:23 κ light chain constant region
SEQ ID NO:24 γ1 heavy chain constant region
SEQ ID NO:25 γ1 heavy chain constant region PVA236/GLPSS331 (IgG1v1)
SEQ ID NO:26 γ1 heavy chain constant region L234A/L235A (IgG1v2)
SEQ ID NO:27 γ4 heavy chain constant region
SEQ ID NO:28 γ4 heavy chain constant region S228/L235E (IgG4v1)
SEQ ID NO:29-32 Heavy chain CDR1
SEQ ID NO:33-37 Heavy chain CDR2
SEQ ID NO:38-42 Heavy chain CDR3
SEQ ID NO:43-44 Light chain CDR1
SEQ ID NO:45-46 Light chain CDR2
SEQ ID NO:47-52 Light chain CDR3

In one embodiment the present invention provides a formulation wherein the antibody is present in an amount in the range of from 10 to 150 mg/mL, preferably from 10 to 50 mg/mL. The antagonistic monoclonal antibodies against P-selectin may be produced by hybridoma cell lines. The preferred hybridoma cell lines are hu-Mab<P-selectin>LC 1004-001 (antibody HuMab 001) hu-Mab<P-selectin>LC 1004-002 (antibody HuMab 002) and hu-Mab<P-selectin>LC 1004-017 (antibody HuMab 017), which were deposited, under the Budapest Treaty on the international recognition of the deposit of microorganisms for the purposes of patent procedure, with Deutsche Sammlung von Mikroorganismen and Zellkulturen GmbH (DSMZ), Germany:

| Cell line | Deposition No. | Date of Deposit |
|---|---|---|
| hu-Mab<P-selectin>LC 1004-001 | DSM ACC2640 | 30 Mar. 2004 |
| hu-Mab<P-selectin>LC 1004-002 | DSM ACC2641 | 30 Mar. 2004 |
| hu-Mab<P-selectin>LC 1004-017 | DSM ACC2642 | 30 Mar. 2004 |

The antibodies useful in the formulations according to the invention are preferably produced by recombinant means, e.g. by those described in WO2006/072564. Such methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis) by standard techniques, including alkaline/SDS treatment, CsCl banding, column chromatography, agarose gel electrophoresis, and others well known in the art, e.g. as described in WO2006/072564.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers and phosphate-buffers. Still preferred buffers comprise L-histidine or mixtures of L-histidine and L-histidine hydrochloride with pH adjustment with an acid or a base known in the art. The above-mentioned buffers are generally used in an amount of about 1 mM to about 100 mM, preferably of about 5 mM to about 50 mM and more preferably of about 10-20 mM. Independently from the buffer used, the pH can be adjusted at a value comprising about 4.0 to about 7.0 and preferably about 5.0 to about 6.5 and still preferably about 5.5 to about 6.0 with an acid or a base known in the art, e.g. hydrochloric acid, acetic acid, phosphoric acid, sulfuric acid and citric acid, sodium hydroxide and potassium hydroxide.

The term “surfactant” as used herein denotes a pharmaceutically acceptable excipient which is used to protect protein formulations against mechanical stresses like agitation and shearing. Examples of pharmaceutically acceptable surfactants include polyoxyethylensorbitan fatty acid esters (Tween®), polyoxyethylene alkyl ethers (Brij®), alkylphenylpolyoxyethylene ethers (Triton®-X), polyoxyethylene-polyoxypropylene copolymer (Poloxamer™, Pluronic®), and sodium dodecyl sulphate (SDS). Preferred polyoxyethylenesorbitan-fatty acid esters are polysorbate 20, (sold under the trademark Tween® 20) and polysorbate 80 (sold under the trademark Tween® 80). Preferred polyethylene-polypropylene copolymers are those sold under the names Pluronic® F68 or Poloxamer™ 188. Preferred polyoxyethylene alkyl ethers are those sold under the trademark Brij®. Preferred alkylphenolpolyoxyethylene ethers are sold under the tradename Triton-X. When polysorbate 20 (Tween®) and polysorbate 80 (Tween® 80) are used they are generally used in a concentration range of about 0.001 to about 1%, preferably of about 0.005 to about 0.1% and more preferably about 0.01% to about 0.04% w/v (weight/volume).

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit. Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose, most preferred is trehalose.

The term “amino acid” as used herein denotes a pharmaceutically acceptable organic molecule possessing an amino moiety located at α-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Amino acids are generally used in an amount of about 10 to 500 mM, preferably in an amount of about 10 to about 300 mM and more preferably in an amount of about 100 to about 300 mM.

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in an amount of about 10 mM to about 500 mM, preferably in an amount of about 10 to about 300 mM and more preferably in an amount of about 100 to about 300 mM.

A subgroup within the stabilizers are lyoprotectants. The term “lyoprotectant” denotes pharmaceutical acceptable excipients, which protect the labile active ingredient (e.g. a protein) against destabilizing conditions during the lyophilization process, subsequent storage and reconstitution. Lyoprotectants comprise but are not limited to the group consisting of sugars, polyols (such as e.g. sugar alcohols) and amino acids. Preferred lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine or mixtures thereof. Lyoprotectants are generally used in an amount of about 10 to 500 mM, preferably in an amount of about 10 to about 300 mM and more preferably in an amount of about 100 to about 300 mM.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, gluthathion, cysteine, methionine, citric acid, EDTA. Antioxidants can be used in an amount of about 0.01 to about 100 mM, preferably in an amount of about 5 to about 50 mM and more preferably in an amount of about 5 to about 20 mM.

The term “tonicity agents” as used herein denotes pharmaceutically acceptable tonicity agents. Tonicity agents are used to modulate the tonicity of the formulation. The formulation can be hypotonic, isotonic or hypertonic. Isotonicity in general relates to the osmostic pressure relative of a solution usually relative to that of human blood serum. The formulation according to the invention can be hypotonic, isotonic or hypertonic but will preferably be isotonic. An isotonic formulation is liquid or liquid reconstituted from a solid form, e.g. from a lyophilized form and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids, sugars, in particular glucose. Tonicity agents are generally used in an amount of about 5 mM to about 500 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

The compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The term “liquid” as used herein in connection with the formulation according to the invention denotes a formulation which is liquid at a temperature of at least about 2 to about 8° C. under atmospheric pressure.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. The lyophilizate has usually a residual moisture of about 0.1 to 5% (w/w) and is present as a powder or a physical stable cake. The lyophilizate is characterized by a fast dissolution after addition of a reconstitution medium.

The term “reconstituted formulation” as used herein in connection with the formulation according to the invention denotes a formulation which is lyophilized and re-dissolved by addition of reconstitution medium. The reconstitution medium comprise but is not limited to water for injection (WFI), bacteriostatic water for injection (BWFI), sodium chloride solutions (e.g. 0.9% (w/v) NaCl), glucose solutions (e.g. 5% glucose), surfactant, containing solutions (e.g. 0.01% polysorbate 20), a pH-buffered solution (e.g. phosphate-buffered solutions).

The formulations according to the invention have new and inventive properties causing a benefit for a patient suffering from asthma or an allergic disease.

The invention further comprises the use of a formulation according to the invention for the manufacture of a medicament for asthma treatment.

A composition of the present invention can be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

The composition must be sterile and fluid to the extent that the composition is deliverable by syringe. In addition to water, the carrier can be an isotonic buffered saline solution, ethanol, polyol (e.g., glycerol, propylene glycol, and liquid polyetheylene glycol, and the like), and suitable mixtures thereof.

The formulation according to the invention can be administered by intravenous (i.v.), subcutaneous (s.c.) or any other parental administration means such as those known in the pharmaceutical art.

The formulation according to the invention can be prepared by methods known in the art, e.g. ultrafiltration-diafiltration, dialysis, addition and mixing, lyophilization, reconstitution, and combinations thereof. Examples of preparations of formulations according to the invention can be found hereinafter.

### EXAMPLES

### Example 1

### Preparation of Liquid Formulations

huMAb-P-Selectin prepared and fermented and purified as described in WO 2005/100402 was provided at a concentration of approx. 22 to 27 mg/mL in a 20 mM histidine buffer at a pH of approx. 6.0, or in a 20 mM acetate buffer at a pH of approx. 5.2.

For the preparation of the liquid formulations huMAb-P-Selectin the excipients (e.g. trehalose) were added as 2-10-fold stock solutions to the antibody solution. The surfactant was then added as a 2 to 200-fold stock solution or as pure surfactant. Finally the protein concentration was adjusted with a buffer to the final huMAb-P-Selectin concentration of approx. 15 mg/mL.

All formulations were sterile-filtered through 0.22 μm low protein binding filters and aseptically filled under nitrogen atmosphere into sterile 6 mL glass vials closed with ETFE (Copolymer of ethylene and tetrafluoroethylene)-coated rubber stoppers and alucrimp caps. The fill volume was approximately 2.4 mL. These formulations were stored at different ICH climate conditions (5° C., 25° C. and 40° C.) for different intervals of time and stressed by shaking (1 week at a shaking frequency of 200 min⁻¹ at 5° C.) and freeze-thaw stress methods. The samples were analyzed before and after applying the stress tests by the analytical methods 1) UV spectrophotometry, and 2) Size Exclusion Chromatography (SEC).

Size Exclusion Chromatography (SEC) was used to detect soluble high molecular weight species (aggregates) and low molecular weight hydrolysis products (LMW) in the formulations. The method was performed on a Water Alliance® 2795 HPLC instrument equipped with a TOSOH BIOSCIENCE® TSK G3000 SWXL column. Intact monomer, aggregates and hydrolysis products were separated by an isocratic elution profile, using 0.2M K₂HPO₄/KOH, 0.25M KCL, pH 7.0 as mobile phase, and were detected at a wavelength of 280 nm. UV spectroscopy, used for determination of protein content, was performed on a Varian Cary® Bio UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ɛ 〈 \frac{{cm}^{2}}{mg} 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε.

TABLE 1

| | Protein conc. | Size Exclusion - HPLC | | |
|---|---|---|---|---|
| Timepoint | (mg/mL) | HMW (%) | Monomer (%) | LMW (%) |
| | | | 15 mg/mL huMAb-P-Selectin, | |
| | | | 20 mM L-histidine HCl, | |
| | | | 240 mM trehalose, | |
| Formulation A | | | 0.02% polysorbate 20, | |
| Storage at 2-8° C. | | | at pH 6.0 | |
| Initial | 16.7 | 0.2 | 99.8 | 0.0 |
| 5 weeks | 15.8 | 0.2 | 99.8 | 0.0 |
| 11 weeks | 16.2 | 0.3 | 99.7 | 0.0 |
| 23 weeks | 16.6 | 0.4 | 99.0 | 0.6 |
| | | | 15 mg/mL huMAb-P-Selectin, | |
| | | | 20 mM sodium acetate, | |
| | | | 140 mM trehalose, | |
| | | | 75 mM glycine | |
| Formulation B | | | 0.04% polysorbate 20, | |
| Storage at 2-8° C. | | | at pH 5.2 | |
| Initial | 16.5 | 0.1 | 99.9 | 0.0 |
| 5 weeks | 16.1 | 0.2 | 99.8 | 0.0 |
| 11 weeks | 16.0 | 0.2 | 99.8 | 0.0 |
| 26 weeks | 15.6 | 0.1 | 99.9 | 0.0 |

### Example 2

### Preparation of Lyophilized Formulations and Liquid Formulations Reconstituted from Lyophilized Formulations

huMAb-P-Selectin prepared and fermented and purified as described in WO 2005/100402 was provided at a concentration of approx. 22 to 27 mg/mL in a 20 mM histidine buffer at a pH of approx. 6.0, or in a 20 mM succinate buffer at a pH of approx. 5.2, and lyophilized using the freeze-drying cycle reported in Table 2.

TABLE 2
Freeze-drying Cycle type I

| | Shelf | | | Vacuum Set |
|---|---|---|---|---|
| | temperature | Ramp Rate | Hold time | point |
| Step | (° C.) | (° C./min) | (min) | (μbar) |
| Pre-cooling | 5° C. | 0.0 | 60 | — |
| Freezing | −40° C. | 1.0 | 120 | — |
| Primary Drying | −25° C. | 0.5 | 3900 | 80 |
| Secondary Drying | +25° C. | 0.2 | 300 | 80 |

The product was first cooled from room temperature to approx 5° C. (pre-cooling), followed by a freezing step at −40° C. with a plate cooling rate of approx. 1° C./min, followed by a holding step at −40° C. for about 2 hours. The first drying step was performed at a plate temperature of approx. −25° C. and a chamber pressure of approx. 80 μbar for about 65 hours. Subsequently, the second drying step started with a temperature ramp of 0.2° C./min from −25° C. to 25° C., followed by a holding step at 25° C. for at least 5 hours at a chamber pressure of approx. 80 μbar.

Lyophilization was carried out in a Usifroid® SMH-90 LN2 freeze-dryer (Usifroid, Maurepas, France). All lyophilized cakes had a residual water content of about 0.1 to 2.0% as determined by the Karl-Fischer method. The freeze-dried samples were incubated at different temperatures for different intervals of time.

The lyophilized formulations were reconstituted to a final volume of 5.3 mL with water for injection (WFI) yielding an isotonic formulation with an antibody concentration of approx. 15 mg/mL. The reconstitution time of the freeze-dried cakes was below 1 min. Analysis of the reconstituted samples was either performed immediately after reconstitution, or after a 24 hour incubation period of the reconstituted liquid sample at 25° C.

The samples were analyzed by 1) UV spectrophotometry and 2) Size Exclusion Chromatography (SEC).

TABLE 3

| | Protein conc. | Size Exclusion - HPLC | | |
|---|---|---|---|---|
| Timepoint | (mg/mL) | HMW (%) | Monomer (%) | LMW (%) |
| | | | 15 mg/mL huMAb-P-Selectin, | |
| | | | 20 mM L-histidine HCl, | |
| | | | 240 mM trehalose, | |
| Formulation C | | | 0.02% polysorbate 20, | |
| Storage at 2-8° C. | | | at pH 6.0 | |
| Initial | 16.7 | 0.2 | 99.8 | 0.0 |
| 5 weeks | 17.1 | 0.2 | 99.8 | 0.0 |
| 11 weeks | 17.0 | 0.2 | 99.8 | 0.0 |
| 23 weeks | 16.4 | 0.3 | 99.1 | 0.6 |
| | | | 15 mg/mL huMAb-P-Selectin, | |
| | | | 20 mM succinate, | |
| | | | 240 mM trehalose, | |
| | | | 20 mM arginine | |
| Formulation D | | | 0.02% polysorbate 20, | |
| Storage at 2-8° C. | | | at pH 6.0 | |
| Initial | 16.7 | 0.2 | 99.8 | 0.0 |
| 5 weeks | 17.0 | 0.2 | 99.8 | 0.0 |
| 11 weeks | 17.1 | 0.2 | 99.8 | 0.0 |
| 36 weeks | 16.5 | 0.2 | 99.8 | 0.0 |

Unless stated to the contrary, all compounds in the examples were prepared and characterized as described. All ranges recited herein encompass all combinations and subcombinations included within that range limit. All patents and publications cited herein are hereby incorporated by reference in their entirety.

1. A pharmaceutical formulation comprising:
1 to 200 mg/mL of an antibody against P-selectin;
1 to 100 mM of a buffer;
0.001 to 1% of a surfactant; and
10 to 500 mM of a stabilizer wherein the stabilizer is a sugar selected from sucrose and trehalose and wherein said formulation has a pH in the range of from 4.0 to 7.0; and further wherein the antibody against P-selectin comprises the light chain variable domain defined by amino acid sequence SEQ ID No: 3 and the heavy chain variable domain defined by SEQ ID No: 4.

2. A formulation according to claim 1 which is a liquid formulation or a lyophilized formulation or a liquid formulation reconstituted from a lyophilized formulation.

3. A formulation according to claim 1, wherein the antibody concentration is in the range of 10 mg/mL to 150 mg/ml.

4. A formulation according to claim 1, wherein the stabilizer is trehalose.

5. A formulation according to claim 1, wherein the surfactant is polysorbate.

6. A formulation according to claim 1, wherein the buffer is a histidine-buffer.

7. A formulation according to claim 1, selected from the group consisting of:
(1) a formulation having a pH of about 6.0 comprising 1 to 50 mg/mL huMAb-P-selectin, about 20 mM L-histidine HCl, about 240 mM trehalose, and about 0.02% polysorbate 20;
(2) a formulation having a pH of about 5.2 comprising 1 to 50 mg/mL huMAb-P-selectin, about 20 mM sodium acetate, about 140 mM trehalose, about 75 mM glycine, and about 0.04% polysorbate 20;
(3) a formulation having a pH of about 6.0 comprising 1 to 50 mg/mL huMAb-P-selectin, about 20 mM L-histidine HCl, about 240 mM trehalose, and about 0.02% polysorbate 20; and
(4) a formulation having a pH of about 6.0 comprising 1 to 50 mg/mL huMAb-P-selectin, about 20 mM succinate, about 240 mM trehalose, about 20 mM arginine, and about 0.02% polysorbate 20.
